(19) United States
(12) Patent Application Publication
(10) Pub. No.: US 2012/0219480 A1
(43) Pub. Date: Aug. 30, 2012

(54) COMPOSITIONS FOR WET AIR SCRUBBERS AND METHODS FOR OPERATING AND CLEANING WET AIR SCRUBBERS USING THE SAME

(75) Inventors: William E. Simpson, New Berlin, WI (US); Keith W. Kennedy, Lebanon, OH (US); Dale A. Grinstead, Racine, WI (US); Michael F. Coughlin, Cincinnati, OH (US); Kenneth J. Roach, Racine, WI (US); Stephen Lyon, Racine, WI (US); Maynard J. Riley, Racine, WI (US); Garry W. Crossdale, Ripley (GB)

(73) Assignee: DIVERSEY, INC., Sturtevant, WI (US)

(21) Appl. No.: 13/392,995

(22) PCT Filed: Aug. 31, 2010
(86) PCT No.: PCT/US2010/047344

§ 371 Date: May 16, 2012

Related U.S. Application Data
(60) Provisional application No. 61/238,624, filed on Aug. 31, 2009.
(60) Provisional application No. 61/239,325, filed on Sep. 2, 2009.
(60) Provisional application No. 61/311,692, filed on Mar. 8, 2010.

(51) Int. Cl. A61L 9/00

(52) U.S. Cl. 423/210

(57) ABSTRACT

A composition for use in air scrubbers, and particularly, cleaning air scrubbers is provided, along with methods for cleaning air scrubbers using the same. The composition includes at least one of components A, B, C, and D. Component A includes at least one surfactant and at least one enzyme. Component B includes at least one surfactant. Component C includes at least one pH control agent. Component D includes at least one defoamer. The compositions may be used to remove volatile organic compounds from air.

### CROSS-REFERENCE TO RELATED APPLICATIONS

This application claims priority to U.S. Provisional Application Nos. 61/238,624 filed Aug. 31, 2009, 61/239,325 filed Sep. 2, 2009, and 61/311,692 filed Mar. 8, 2010, which are incorporated herein by reference in their entireties.

### FIELD OF INVENTION

The present invention relates to compositions for cleaning and operating wet air scrubbers.

### INTRODUCTION

Wet air scrubbers may be used with rendering plants. Rendering plants process unwanted and unused animal parts and tissue, for example, from meat-processing houses and slaughter houses, and convert them into useful finished goods including animal feed, fuel oil, and Pharma Cos ingredients. The air surrounding rendering plant equipment may have a bad odor and may contain volatile organic compounds (VOCs).

Air scrubbers may be used to reduce or eliminate malodor at rendering plants, for example, by removing volatile organic compounds from the air. A wet air scrubber operates on the principle that VOCs in the air diffuse into water and consequently are prevented from entering the atmosphere. Air scrubbers may, for example, comprise a tower with water flowing from the top of the tower to the bottom of the tower, with water then recycled to the top of the tower again. As air from a rendering plant is flowed through the air scrubber, VOCs may be removed from the air. While some air scrubbers rely on sprayed water to create an air/water interface for purification, some air scrubbers use plastic or stainless steel media to increase air/water surface area and to decrease water flow as the air flows upwards through the scrubber. If the water in the air scrubber is being treated with an oxidizer or other conventional treatment solution including acidified bleach, chlorine dioxide, ozone and/or permanganates, this media is often fouled with insoluble high molecular weight proteins, oils, greases, and other organic debris. This fouling may cause several problems. First, as the media is fouled, the cross section available for air flow decreases, and the removal efficiency of volatile organic compounds is diminished. Second, the organic debris may act as a source of odor, especially after downtime when the water is not recirculating, if the recirculating water temperature temporarily increases during operation due to process temperature changes, and/or when treatment chemical is not being fed. The air scrubber at rest may allow hydrogen sulfide generating anaerobic bacteria under the deposits to flourish.

Other aspects of the invention will become apparent by consideration of the detailed description and accompanying drawings.

### SUMMARY

In certain embodiments, methods are provided of operating a wet air scrubber, the methods including (a) adding to the wet air scrubber a composition comprising water and at least one of Component A and Component B, (b) adding a pH control agent to the composition to maintain a pH of about 6 to about 9, and (c) maintaining the water temperature at less than about 50° C. Component A may comprise (1) an amphoteric surfactant and (2) an enzyme. Component B may comprise at least one of (1) a detersive surfactant and a defoamer; (2) at least one low-foam surfactant selected from a capped alkoxylate, an EO/PO (ethylene oxide group/propylene oxide group) block copolymer, a fatty alcohol alkoxylate, a nonionic ethylene oxide (EO) containing surfactant, an alkoxylated amine, an amine surfactant, a low cloud point nonionic surfactant, and a combination thereof; and (3) a combination of (1) and (2).

### BRIEF DESCRIPTION OF THE DRAWINGS

FIG. 1 is a schematic diagram of a wet air scrubber.

FIG. 2 is a graph of the removal of contaminants from air using an air scrubber operated with a composition according to Example 1 and conventional cleaners.

FIG. 3 is a graph of ammonia reduction in a wet air scrubber using a composition according to Example 1, as a function of pH.

FIG. 4 is a graph of hydrogen sulfide reduction in a wet air scrubber using a composition according to Example 1, as a function of pH.

FIG. 5 is a graph of A6 soil removed (% clean) for various low foaming surfactants.

FIG. 6 is a graph of foam height for compositions comprising various low-foam surfactants and ReNew A at 30° C.

FIG. 7 is a graph of foam height for compositions comprising various low-foam surfactants and ReNew A at 30° C.

FIG. 8 is a graph of foam height for compositions comprising 60 ppm Triton™ DF-12 and 170 ppm ReNew A at various temperatures.

FIG. 9 is a graph of foam height for compositions comprising 60 ppm Plurafac® LF-403 and 170 ppm ReNew A at various temperatures.

FIG. 10 is a graph of foam height for compositions comprising 60 ppm Plurafac® LF-101 and 170 ppm ReNew A at various temperatures.

FIG. 11 is a graph of foam height at 30° C. for compositions comprising 30 ppm of a low foam surfactant (Plurafac® LF-403, Triton™ DF-12, or Plurafac® LF-101), 30 ppm Neodol™ 23-6.5, and 170 ppm ReNew A.

FIG. 12 is a graph of foam height at 12° C. for compositions comprising 170 ppm of various low foam surfactants.

FIG. 13 is a graph of % soil removal for various surfactant compositions, measured at 0.5% and 1% surfactant.

FIG. 14 is a statistical analysis of the data presented in FIG. 13.

FIG. 15 is a graph of % soil removal for compositions comprising various ratios of ReNew A and Triton™ DF-12, measured at 0.7% surfactant, at 20° C. and 40° C.

FIG. 16 is a statistical analysis of the data presented in FIG. 15.

### DETAILED DESCRIPTION

Before any embodiments of the invention are explained in detail, it is to be understood that the invention is not limited in its application to the details of construction and the arrangement of components set forth in the following description or illustrated in the following drawings. The invention is capable of other embodiments and of being practiced or of being carried out in various ways.

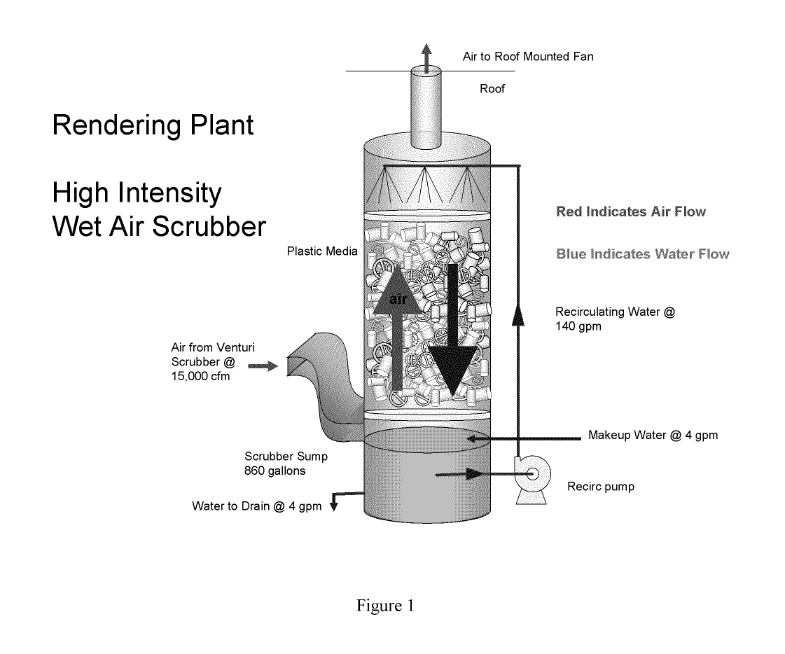

In certain embodiments, provided is a composition for use in a wet air scrubber, for example, an air scrubber at a rendering plant as depicted schematically in FIG. 1. Wet air scrubbers may include, for example, a packed tower scrubber, a spray tower scrubber, an orifice scrubber, a venturi scrubber, a fiber-bed scrubber, an impingement-plate scrubber, a spray nozzle scrubber, a fluidized-bed scrubber, a packed-bed scrubber, multiple-stage scrubbers, baffle spray scrubber, a counter-flow scrubber, a crossflow scrubber, and combinations thereof. Wet air scrubbers can be custom designed by, for example, an independent designer, or competent staff at a rendering faciloity. Wet air scrubbers are commercially available, for example, from Verantis, AC Corporation, Scp Control Inc., and Millpoint Industries Inc. Compositions according to the invention may also be used with packed media wet air scrubbers at factories or plants including, but not limited to, municipal wastewater plants, pet food plants, flavor and fragrance plants, breweries, and grain operations such as corn processing. The compositions according to the invention may remediate odors and provide air scrubbers with more effective pollution control. The compositions may be at least one of oxidizer-free, non-bacterial, and pH neutral, but do not act solely as perfumes or masking agents. The compositions may comprise one or more of the following components: A, B, C, and D.

Component A may comprise at least one surfactant and at least one enzyme. The surfactant may include, but not be limited to, a non-ionic surfactant, a cationic surfactant, an anionic surfactant, an amphoteric surfactant, or combinations thereof.

Examples of nonionic surfactants include, but are not limited to, amides, alkanolamides, amine oxides, block polymers, alkoxylated primary and secondary alcohols, alkoxylated alkylphenols, alkoxylated fatty esters, sorbitan derivatives, glycerol esters, propoxylated and alkoxylated fatty acids, alcohols, and alkyl phenols, glycol esters, polymeric polysaccharides,

Nonionic surfactants are conventionally produced by condensing ethylene oxide with a hydrocarbon having a reactive hydrogen atom, e.g., a hydroxyl, carboxylic acid group, primary and secondary amino, or primary or secondary amido group, in the presence of an acidic or basic catalyst. Nonionic surfactants may have the general formula RA(CH₂CH₂O)ₙH wherein R represents the hydrophobic moiety, A represents the group carrying the reactive hydrogen atom and n represents the average number of ethylene oxide moieties. R may be a primary or a secondary, straight or slightly branched, aliphatic alcohol having from about 8 to about 24 carbon atoms. A more complete disclosure of nonionic surfactants can be found in U.S. Pat. No. 4,111,855, Barrat, et al., issued Sep. 5, 1978, and U.S. Pat. No. 4,865,773, Kim et al., issued Sep. 12, 1989, which are hereby fully incorporated by reference.

Other nonionic surfactants useful in the composition include ethoxylated alcohols or ethoxylated alkyl phenols of the formula R(OC₂H₄)ₙOH, wherein R is an aliphatic hydrocarbon radical containing from about 8 to about 18 carbon atoms or an alkyl phenyl radical in which the alkyl group contains from about 8 to about 15 carbon atoms, and n is from about 2 to about 14. Examples of such surfactants are listed in U.S. Pat. No. 3,717,630, Booth, issued Feb. 20, 1973, U.S. Pat. No. 3,332,880, Kessler et al., issued Jul. 25, 1967, and U.S. Pat. No. 4,284,435, Fox, issued Aug. 18, 1981, which are hereby fully incorporated by reference.

Moreover, other nonionic surfactants include the condensation products of alkyl phenols having an alkyl group containing from about 8 to about 15 carbon atoms in either a straight chain or branched chain configuration with ethylene oxide, said ethylene oxide being present in an amount from about 2 to about 14 moles of ethylene oxide per mole of alkyl phenol. The alkyl substituent in such compounds can be derived, for example, from polymerized propylene, diisobutylene, and the like. Examples of compounds of this type include nonyl phenol condensed with about 9 moles of ethylene oxide per mole of nonyl phenol, dodecyl phenol condensed with about 8 moles of ethylene oxide per mole of phenol, and the commercially available T-DET® 9.5 marketed by Harcros Chemicals Incorporated.

Other useful nonionic surfactants are the condensation products of aliphatic alcohols with from about 2 to about 14 moles of ethylene oxide. The alkyl chain of the aliphatic alcohol can either be straight or branched, primary or secondary, and may contain from about 8 to about 18 carbon atoms. Examples of such ethoxylated alcohols include secondary alcohol nonionic surfactants such as ENS-70, the condensation product of myristyl alcohol condensed with about 9 moles of ethylene oxide per mole of alcohol, and the condensation product of about 7 moles of ethylene oxide with coconut alcohol (a mixture of fatty alcohols with alkyl chains varying in length from 10 to 14 carbon atoms). Examples of commercially available nonionic surfactants in this type include: Tergitol™ 15-S-7 or 15-S-9 marketed by Union Carbide Corporation; Neodol™ 45-9, Neodol™ 23-6.5, Neodol™ 45-7 and Neodol™ 45-4 marketed by Shell Chemical Company; Kyro EOB marketed by The Procter & Gamble Company; and Berol® 260 and Berol® 266 marketed by Akzo Nobel. Other suitable non-ionic surfactants include Neodol™ ethoxylates, commercially available from Shell Chemicals (Houston, Tex.) and Tergitol™ surfactants, commercially available from Dow (Midland, Mich.). A mixture of nonionic surfactants may also be used.

Examples of anionic surfactants include, but are not limited to, sulfosuccinates and derivatives, sulfates of ethoxylated alcohols, sulfates of alcohols, sulfonates and sulfonic acid derivatives, sulfates and sulfonates of alkoxylated alkylphenols, phosphate esters, and polymeric surfactants. Suitably, anionic surfactants may include, but are not limited to, alkyl sulfate, ether sulfate, alkyl benzene sulfonate, alpha olefin sulfonate, diphenyloxide disulfonate, alkyl naphthalene sulfonate, sulfosuccinate, sulfosuccinamate, naphthalene-formaldehyde condensate, isethionate, N-methyl taurate, phosphate ester, and ether carboxylate.

Amphoteric surfactants may include betaines and betaine derivatives. Amphoteric surfactants may also include, but are not limited to, amphoteric imadazoline derivatives and fatty amine and fatty amine ethoxylate derivatives. Amphoteric imadazoline derivatives may include, but are limited to, amphodiacetates, amphoacetates, amphocarboxylates, amphopropionate, amphodipropionate, and hydroxypropyl sulfonate. Fatty amine and fatty amine ethoxylate derivatives may include, but are not limited to, betaines, alkyl betaine, sultaine, dihydroxyethyl glycinate, alkyl amidopropyl betaine, and aminopropionate.

Cationic surfactants may include amine surfactants, those containing non-quaternary nitrogen, those containing quaternary nitrogen bases, those containing non-nitrogenous bases and combinations thereof. Such surfactants are disclosed in U.S. Pat. No. 3,457,109, Peist, issued Jul. 22, 1969, U.S. Pat. No. 3,222,201, Boyle, issued Dec. 7, 1965 and U.S. Pat. No. 3,222,213, Clark, issued Dec. 7, 1965, which are hereby fully incorporated by reference.

One category of cationic surfactants may include quaternary ammonium compounds with the general formula RXYZ N⁺A⁻, wherein R is an aliphatic or cycloaliphatic group having from 8 to 20 carbon atoms and X, Y and Z are members selected from the group consisting of alkyl, hydroxylated alkyl, phenyl and benzyl. A⁻ is a water soluble anion that may include, but is not limited to, a halogen, methosulfate, ethosulfate, sulfate and bisulfate. The R group may be bonded to the quaternary group through hetero atoms or atom groups such as —O—, —COO—, —CON—, —N—, and —S—. Examples of such compounds include, but are not limited to, trimethyl-hexadecyl-ammonium sulfate, diethyl-octadecyl-phenyl-ammonium sulfate, dimethyl-dodecyl-benzyl-ammonium chloride, octadecylamino-ethyl-trimethyl-ammonium bisulfate, stearylamido-ethyl-trimethyl-ammonium methosulfate, dodecyloxy-methyl-trimethyl-ammonium chloride, cocoalkylcarboxyethyl-di-(hydroxyethyl)-methyl-ammonium methosulfate, and combinations thereof.

Another category of cationic surfactants may be of the di-long chain quaternary ammonium type having the general formula XYRR₁N⁺A⁻, wherein X and Y chains may contain an average of from about 12 to about 22 carbon atoms and R and R₁ may be hydrogen or C1 to C4 alkyl or hydroxyalkyl groups. Although X and Y may contain long chain alkyl groups, X and Y may also contain hydroxy groups or may contain heteroatoms or other linkages, such as double or triple carbon-carbon bonds, and ester, amide, or ether linkages, as long as each chain falls within the above carbon atom ranges.

An additional category of cationic surfactant may include the bis(ethoxylated) ammonium quaternary compounds having the general formula:

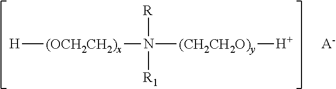

wherein R is methyl, ethyl or propyl group, R₁ is an alkyl group having from 8 to 18 carbon atoms, an alkenyl group having 8 to 18 carbon atoms or mixtures thereof, x is a number from 1 to 40, y is a number from 1 to 40, wherein x+y is between 10 to 60, and A is a water soluble anion. Examples of such compounds include, but are not limited to, alkyl bis(ethoxy) methyl ammonium methyl sulfate (15 moles EO), stearyl methyl bis(ethoxy) ammonium chloride (12 moles EO), stearyl ethyl bis(ethoxy) ammonium ethyl sulfate (15 moles EO), tallow methyl bis(ethoxy) ammonium methyl sulfate (15 moles EO), tallow ethyl bis(ethoxy) ammonium ethyl sulfate (15 moles EO), hydrogenated tallow methyl bis(ethoxy) ammonium chloride (15 moles EO), coco methyl bis(ethoxy) ammonium methyl sulfate (20 moles EO), and combinations thereof.

Other cationic surfactants may include sulfonium, phosphonium, and mono- or tri-long chain quaternary ammonium materials and those described in U.S. Pat. No. 4,259,217, Murphy, issued Mar. 31, 1981, U.S. Pat. No. 4,222,905, Cockrell, Sep., 16, 1980, U.S. Pat. No. 4,260,529, Letton, issued Apr. 7, 1981, U.S. Pat. No. 4,228,042, Letton, issued Oct. 14, 1980, and U.S. Pat. No. 4,228,044, Cushman, issued Oct. 14, 1980, each of which is fully incorporated herein by reference.

Additional cationic surfactants may include ditallowalkyldimethyl (or diethyl or dihydroxyethyl) ammonium chloride, ditallowalkyldimethylammonium methyl sulfate, dihexadecylalkyl (C16) dimethyl (or diethyl, or dihydroxyethyl) ammonium chloride, dioctodecylalkyl (C18) dimethylammonium chloride, dieicosylalkyl (C20) dimethylammonium chloride, methyl (1) tallowalkyl amido ethyl (2) tallowalkyl imidazolinium methyl sulfate (commercially available as Varisoft 475 from Ashland Chemical Company), or mixtures of those surfactants.

Additional surfactants that may be useful according to the invention may be found in U.S. Pat. Nos. 6,054,139, 6,547,063, and 7,572,933, each of which is incorporated herein by reference in their entireties.

Suitably, the surfactant of component A may be amphoteric. The surfactant of component A may be a zwitterion. Component A may comprise two or more surfactants. Component A may comprise at least about 1%, at least about 2%, at least about 5%, or at least about 7% of surfactant by weight. Component A may also comprise less than about 95%, less than about 15%, less than about 10%, less than about 8% of surfactant by weight. Component A may comprise about 1 to about 95 wt %, about 2 to about 15 wt %, about 5 to about 15 wt %, about 5 to about 10 wt %, or about 7 to about 8 wt % of surfactant.

The enzyme of component A may be at least one of a protease, a lipase, a hydrolase, a cellulase, an amylase, and a combination thereof. Component A may comprise an amylase and a protease. Component A may comprise less than about 10 wt %, less than about 5 wt %, less than about 2 wt %, less than about 1 wt %, or less than about 0.5 wt % of enzyme. There are many materials derived from plant, animal, and microbial sources that have been known to those skilled in the art to be rich sources of enzymes. These source materials may be used with full, partial, or no purification of enzymes to obtain enzymes for use in the composition. For example, the enzyme may be exogenously produced from a recombinant or wild-type organism. The enzyme may be purified. The enzyme may be a fermentation product.

The balance of component A may or may not comprise preservatives, fragrances, and enzyme stabilizers such as propylene glycol and borates.

Suitably, component A is commercially available as ReNew A (Diversey, Sturtevant, Wis.).

Component B may comprise at least one surfactant. The surfactant may include, but not be limited to, a non-ionic surfactant, a cationic surfactant, an anionic surfactant, a low-foam surfactant, or a combination thereof. The at least one surfactant of component B may be a detersive surfactant. Examples of surfactants are provided above with respect to component A.

In certain embodiments, component B may comprise a low-foam surfactant. Low-foam surfactants may include, but are not limited to, nonionic ethylene oxide (EO) containing surfactants, capped alkoxylates such as capped ethoxylates, capped alcohol alkoxylates such as capped alcohol ethoxylates, EO/PO (ethylene oxide group/propylene oxide group block copolymer, fatty alcohol alkoxylates, alkoxylated amines such as ethoxylated amines, amine surfactants, low cloud point nonionic surfactants (as detailed below), and those set forth above with respect component A. Suitably, low-foam surfactants may include, but are not limited to, Triton™ surfactants such as Triton™ DF-12, Triton™ CF-32 and Triton™ CF-10 (Dow, Midland, Mich.); Pluronic® surfactants such as Pluronic® 25-R-2, Pluronic® N-3, Pluronic® SLF-18, Pluronic® L-61, and Pluronic® L-62 (BASF, Florham Park, N.J.); and Plurafac® surfactants such as Plurafac® LF-403, Plurafac® LF-101, and Plurafac® LF-400 (BASF, Florham Park, N.J.).

In certain embodiments, the surfactant of component B may be non-ionic. Suitably, the surfactant of component B may be selected from the group consisting of a primary alcohol ethoxylate and a secondary alcohol ethoxylate. The surfactant of component B may be an alkyl phenol, although alkyl phenol surfactants may degrade to non-environmentally-friendly compounds, which may not be desired in certain applications. The surfactant of component B may have a hydrophilic-lipophilic balance (HLB) of at least about 8, at least about 9.5, at least about 10, or at least about 11.2. The surfactant of component B may have a HLB of less than about 14, less than about 12.5, less than about 12, or less than about 11.8. The surfactant of component B may have a HLB of about 8 to about 14, about 9.5 to about 12.5, about 10 to about 12, and about 11.2 to about 11.8. Component B may comprise any combination of two or more non-ionic surfactants combined in a ratio to achieve an HLB of at least about 8, at least about 9.5, at least about 10, or at least about 11.2. Component B may comprise any combination of two or more non-ionic surfactants combined in a ratio to achieve an HLB of less than about 14, less than about 12.5, less than about 12, or less than about 11.8. Component B may comprise any combination of two or more non-ionic surfactants combined in a ratio to achieve an HLB of about 8 to about 14, about 9.5 to about 12.5, about 10 to about 12, and about 11.2 to about 11.8. HLB of a surfactant is a measure of the degree to which it is hydrophilic or lipophilic, determined by calculating values for the different regions of the molecule. HLB values may be found, for example, in McCutcheon's Emulsifiers & Detergents (2009, MC Publishing Company, Glen Rock, N.J.). For example, component B may include Tergitol™ 15-S-7, commercially available from Dow (Midland, Mich.). Component B may include at least one of Neodol™ 91-6, Neodol™ 91-2.5, Neodol™ 23-6.5, Neodol™ 23-3, Neodol™ 45-7, Neodol™ 45-4, Neodol™ 25-3, Neodol™ 25-12, Neodol™ 25-9, Neodol™ 91-8, Neodol™ 25-5, Neodol™ 25-7 (commercially available from Shell Chemicals, Houston, Tex.); Triton™ surfactants such as Triton™ DF-12, Triton™ CF-32, Triton™ CF-10 (Dow, Midland, Mich.); Plurafac® LF-403 (BASF, Florham Park, N.J.), Pluronic® surfactants such as Pluronic® 25-R-2, Pluronic® N-3, Pluronic® SLF-18, Pluronic® L-62 (BASF, Florham Park, N.J.); or a combination thereof. Component B may comprise at least two surfactants combined in any ratio including, but not limited to, 1:1, 1:2, 1:3, 1:4, 1:5, 1:6, 1:7, 1:8, 1:9, 1:10, 1:15, 15:1, 10:1, 9:1, 8:1, 7:1, 6:1, 5:1, 4:1, 3:1, or 2:1. Component B is commercially available as ReNew B (Diversey, Sturtevant, Wis.). Component B may comprise Triton™ DF-12, Plurafac® LF-403, or a combination thereof.

Component C may comprise a pH control agent. The pH control agent may comprise an acid, a base, or a combination thereof. Component C may comprise an acid such as mineral acids, organic acids, or any other acid effective to drive ammonia to ammonium in the air scrubber. Component C may comprise an alkaline metal salt. Suitably, component C may comprise citric acid. Suitably, component C is commercially available as ReNew Balance (Diversey, Sturtevant, Wis.). Component C may be added to the air scrubber in an amount effective to maintain the pH of the solution in the air scrubber at a pH effective to eliminate malodor compounds such as H₂5 as well as NH₃. With a pKa of 9.3 for the reaction NH₃+H⁺→NH₄⁺, a pH of about 7.3 may drive the reaction to about 99% NH₄⁺. As such, NH₃ may be one malodorous compound formed in a rendering plant that may be converted at low pH to NH₄⁺, thus converting malodor or noxious gas to a water soluble salt that may not evaporate or smell bad but may remain in the recirculating water. H₂S may be eliminated at high pH. H₂S may not be a factor for some air scrubbers, as H₂S is a metabolic by-product of anaerobic bacteria and many air scrubbers are fully aerated. As such, the pH of an air scrubber may be balanced between about 7.5 and lower for controlling NH₃ levels, and about 7.5 and higher for controlling H₂5 levels. Controlling NH₃ levels includes controlling low molecular weight organic nitrogen-containing compounds like amines and amine oxides, and controlling H₂5 levels also includes controlling low molecular weight sulfur-containing compounds like mercaptans and thiols. The pH of the air scrubber may be maintained at a pH of at least about 5.0, at least about 6.0, at least about 6.8, at least about 7.0, at least about 7.3, or at least about 7.5. The pH of the air scrubber may be maintained at a pH of less than about 9.0, less than about 8.0, less than about 7.9, less than about 7.8, or less than about 7.6. Suitably, the pH of the air scrubber is maintained at a pH of about 5.0 to about 9.0, about 6.0 to about 9.0, about 6.8 to about 8.0, about 7.0 to about 7.9, about 7.3 to about 7.8, and about 7.5 to about 7.6. Suitably, component C is added to the air scrubber in amount effective to maintain the pH of the air scrubber at about 7.5-7.6.

Compositions according to the invention may include component D. Component D may comprise a chemical defoamer. Examples of chemical defoamers include, but are not limited to, silicone-based defoamers; steric acids or fatty acids such as long chain fatty acids; fatty alcohols; oils such as paraffin, mineral oil, and vegetable oil; ethoxylated nonionic surfactants used above their cloud point, EO/PO (ethylene oxide group/propylene oxide group) block copolymers; and low-foaming surfactants such as capped ethoxylates. Suitably, the defoamer may be hydrophobic silica. In certain embodiments, component D may comprise at least one of Antifoam SE-21 (Dow Corning), Silfoam SD860, Silfoam SD168 (Wacker Corp), Tego 3062 (Goldschmidt), or a combination thereof. Many nonionic ethylene oxide derivative surfactants, as described above for component B, are water soluble and have cloud points below the intended use temperature of the composition (as described below), and therefore may be useful defoaming agents. As the temperature is increased, the cloud point may be reached, at which point the surfactant precipitates out of solution and may function as a defoamer. The surfactant may act as a defoamer when used at temperatures at or above this cloud point. The cloud point of nonionic surfactants is defined as the temperature at which a 1 wt % aqueous solution becomes insoluble in water. Suitably, a chemical defoamer of component D may comprise surfactants having cloud points that are below the intended use temperature of the air scrubber composition. Examples of ethylene oxide derivatives include, but are not limited to, polyoxyethylene-polyoxypropylene block copolymers, alcohol alkoxylates (e.g., Triton™ DF-12), low molecular weight EO containing surfactants, and derivatives thereof. Some examples of polyoxyethylene-polyoxypropylene block copolymers include, but are not limited to, the following:

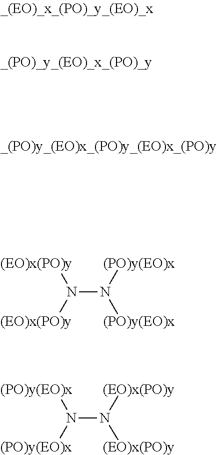

wherein EO represents an ethylene oxide group, PO represents a propylene oxide group, and x and y reflect the average molecular proportion of each alkylene oxide monomer in the overall block copolymer composition. Some examples of block copolymer surfactants include, but are not limited to, commercial products such as Pluronic® surfactants such as Pluronic® 25-R-2 and TETRONIC® surfactants (BASF, Florham Park, N.J.). It is believed that one skilled in the art would understand that a nonionic surfactant with an unacceptably high cloud point temperature or an unacceptably high molecular weight would either produce unacceptable foaming levels or fail to provide adequate defoaming capacity in a detersive or cleaning composition. Other examples of defoamers include Plurafac® alcohol alkoxylates such as Plurafac® LF-403 (BASF, Florham Park, N.J.) and Triton™ surfactants such as Triton™ DF-12 (Dow, Midland, Mich.). Component D may be added to the other components in combination with or separately. Component D may be added to the air scrubber composition to a final concentration of at least about 0.1%, at least about 0.5%, at least about 1%, or at least about 3% (wt %). Component D may be added to the air scrubber composition to a final concentration of less than about 5%, less than about 3%, or less than about 1%. The concentration of component D may be about 25 to about 400 ppm, and may be about 25-200 ppm.

A mechanical defoamer may be used in conjunction with the compositions or as an alternative to component D. Mechanical defoamers are known by those of skill in the art and may include, but are not limited to, circulating disks, ultra sound, pulsed lasers, fluidized beds, high pressure spray nozzles. Examples of mechanical defoamers are described in Takesono et al., Journal of Chemical Technology & Biotechnology (2002) 78: 48-55; Deshpande et al. Chemical Engineering and Processing (2000) 39: 207-217; and U.S. Pat. Nos. 6,599,948; 6,962,618; and 5,299,175, each of which is fully incorporated by reference.

As discussed above, the air scrubber composition may comprise at least one of components A, B, C, and D. The components may be pre-mixed or added individually or in any combination. The air scrubber composition may comprise component A. The air scrubber composition may comprise component B. Component B may comprise at least one of capped alkoxylate, an EO/PO (ethylene oxide group/propylene oxide group) block copolymer, a fatty alcohol alkoxylate, a nonionic ethylene oxide (EO) containing surfactant, an alkoxylated amine, an amine surfactant, a low cloud point nonionic surfactant, and a combination thereof. The air scrubber composition may comprise component B and component D. The air scrubber composition may comprise components A, B, C, and D. The air scrubber composition may comprise component A, component B comprising a low-foaming surfactant, and component C. The air scrubber composition may comprise components B, C, and D. The air scrubber composition may comprise components A, C, and D. The air scrubber composition may comprise component A and component B comprising a low-foaming surfactant. Additional variations and combinations are envisioned.

The air scrubber composition may optionally include additional ingredients. Additional ingredients may include, but are not limited to, chelating agents, dispersants, corrosion inhibitors, fragrance or odor counteractants, enzymes, bacteria, anti-microbial agents, acid, alkaline metal salts, tracing compounds, hydrotropes, solvents, viscosity modifiers, and appearance modifiers known to those skilled in the art. Examples of chelants include, but are not limited to, EDTA, NTA, and poly phosphates. Examples of dispersants include, but art not limited to, acrylic acid homopolymers, polymers of acrylic and maleic acids and hydrophobically modified versions of these polymers. Examples of corrosion inhibitors include, but are not limited to, nitrite salts, silicate and polysilicate salts, and mercaptobenzothiazole (MBT). Examples of odor counteractants include, but are not limited to, cyclodextrins. Examples of hydrotropes include, but are not limited to, sodium xylene sulfonate and sodium cumene sulfonate. Examples of solvents include, but are not limited to, high boiling gylcol ethers. Examples of viscosity modified include, but are not limited to, natural gums such as guar and synthetic poilymeric resins such as carboxy methyl cellulose and Carbopol brand polymers.

The air scrubber composition may be used in the air scrubber at an operating temperature of at least about 10° C., or at least about 15° C. The air scrubber composition may be used in the air scrubber at an operating temperature of less than about 80° C., or less than about 45° C. The air scrubber composition may be used in the air scrubber at an operating temperature of about 10° C. to about 80° C., and about 15° C. to about 45° C. In certain embodiments, the operating temperature may be less than about 50° C. In certain embodiments, the operating temperature may be below about 40° C. The operating temperature may be on at which the malodor is minimized. Lower temperature increases solubility of volatile components.

In certain embodiments, provides are methods of operating an air scrubber. The methods may comprise (a) adding to the wet air scrubber a composition comprising water and at least one of Component A and Component B as described above, (b) adding a pH control agent to the composition to maintain the pH as described above, and (c) maintaining the water temperature in the air scrubber as described above.

Components A and B may be added to the air scrubber individually or in combination. The target concentration for component A and component B may be determined based on the Chemical Oxygen Demand (COD, mg/L) of the recirculating water in the air scrubber. COD is a measure of how much organic material is entering the water, and is an indication of the VOC level. The COD of the recirculating water may be determined by on-site measurement or by sending a sample to an off-site laboratory. As described below, the COD levels may depend on the organic loading of the air coming into the air scrubber, and this loading may fluctuate over time based on what raw materials are running through the cooker and include VOCs and aerosolized organic materials such as fat, oil, grease, bone meal, and protein. Once a representative value is determined, the following equations may be used to calculate the target concentrations of components A and B for each air scrubber:

[Component A]=(0.075)(COD in mg/L)+125=mg/L of Component A

[Component B]=(0.070)(COD in mg/L)+25=mg/L of Component B

The concentration of COD in the recirculating water of a wet air scrubber is typically less than about 1,000 mg/L, but compositions according to the invention may be used with any COD concentration. For example, components A and B may be added to a final combined concentration of at least about 1 ppm, at least about 10 ppm, or at least about 100 ppm. Components A and B may be added to a final combined concentration of less than about 750 ppm, less than about 400 ppm, or less than about 300 ppm. Components A and B may be added to a final combined concentration of about 1 ppm to about 750 ppm, about 1 to about 400 ppm, about 10 to about 300 ppm, or about 100 to about 300 ppm. Component A may be added to a final concentration of at least about 50 mg/L, at least about 100 mg/L, or at least about 125 mg/L. Component A may be added to a final concentration of less than about 400 mg/L, less than about 300 mg/L, or less than about 250 mg/L. Component A concentrations may range from about 50 to about 400 mg/L, about 100 to about or about 300 mg/L, or about 125 to about 250 mg/L. Component B may be added to a final concentration of at least about 10 mg/L, at least about 20 mg/L, or at least about 25 mg/L. Component B may be added to a final concentration of less than about 200 mg/L, less than about 95 mg/L, or less than about 75 mg/L. Component B concentrations may range from about 10 to about 200 mg/L, about 20 to about 95 mg/L, or about 25 to about 75 mg/L. The ratio of component A to component B may be at least about 1:1, at least about 1:2, at least about 1:3, at least about 1:4, at least about 1:5, at least about 1:6, at least about 1:7, or at least about 1:8. The ratio of component A to component B may be less than about 1:1, less than about 1:2, less than about 1:3, less than about 1:4, less than about 1:5, less than about 1:6, less than about 1:7, or less than about 1:8. The ratio of component A to component B may be about 1:1, about 1:2, about 1:3, about 1:4, about 1:5, about 1:6, about 1:7, or about 1:8. In certain embodiments, the ratio of component A to component B may be about 1:2 to about 1:6. In certain embodiments, the ratio of component A to component B may be about 1:4.

Component A and component B may be continually added to the recirculating water in the air scrubber to maintain the appropriate concentration, which is further detailed below. The volume of component A and component B to be delivered may be determined as follows:

Hourly Component A Dosage=(Target)(MU Rate)(0.228)=mL/h Component A

Hourly Component B Dosage=(Target)(MU Rate)(0.228)=mL/h Component B

wherein Target=Target concentration of component in mg/L, and MU Rate=Fresh water supply (Make Up) in gallons per hour. The concentrations of components A and B may be monitored by taking measurements of the volume of composition delivered and the volume of make up water being supplied to the air scrubber, and then calculating the maintenance dose as follows:

$$\begin{matrix}Component A Concentration = \frac{( Component A Volume ) ( 4.39 )}{( Make Up Rate )} \\ = mg / L Component A\end{matrix}$$

wherein Component A Volume=Milliliters of Component A delivered to the air scrubber in 1 hour, and Make up Rate=Volume of make up water to the air scrubber in gallons per minute. The concentration of component B may be calculated in the same way.

In certain embodiments, the air scrubber compositions may be low-foam. In a Venturi Scrubber, for example, about 6 inches of foam or less at the bottom of the air scrubber may be low foam and suitable for normal operation, about 6 to about 18 inches of foam at the bottom of the air scrubber may be moderate foam for normal operation, and about 18 to about 72 inches of foam or more at the bottom of the air scrubber may be high foam for normal operation. Too much foam may interfere with operations of the air scrubber. For example, high foam may result in composition leaking out of outlet ducts, build-up of foam around the drain, and damage to equipment including electrical equipment.

The compositions may be added to recirculating water in an air scrubber. The composition may be added continuously to the air scrubber to maintain a concentration of at least one of the components A, B, and C as described above. The compositions may be used in a much less amount than conventional air scrubber treatment solutions and still effectively remove malodor. The compositions may also keep the media of the air scrubbers cleaner than with conventional air scrubber treatment solutions, that is, compositions may be used in air scrubbers without the need for periodic off-line alkaline detergent cleaning to remove grease. Media in air scrubbers may be cleaned during operation (on-line) with compositions according to the invention while simultaneously removing odor from air of a rendering plant. Not to be limited to theory, the mechanism of the compositions according to the invention is thought to be micelle formation in the bulk solution, and the diffusion of the volatile organic compounds into the water is also thought to be enhanced by surface tension reductions at the air/water droplet interface.

To determine how much of the composition to add to the air scrubber during operation, a variety of mechanisms known in the art may be used. For example, a pH detector may be used to monitor the pH of the solution in the air scrubber. The pH of the solution in the air scrubber may be maintained at suitably ranges as detailed above. For example, if the pH goes below about 7.5-7.6, less component C may be added to the air scrubber, and if the pH goes above about 7.5-7.6, more component C may be added to the air scrubber.

Alternatively or in combination with a pH monitoring system, another embodiment of the invention is to monitor the concentration of NH₃ in the air scrubber with an ion-selective electrode. The organic load in an air scrubber as indicated by the NH₄⁺ concentration may vary with time due to fluctuations in raw material composition (e.g. relative fat and protein content, percent moisture, presence of hair or feathers) and raw material availability in the rendering plant. As the raw material changes in the rendering plant from hour to hour, day to day, or season to season, the concentration and composition of gases entering the air scrubber may change. The primary organic compound entering the air scrubber may be ammonia (NH₃), and this gas may be used as a marker to determine the air scrubber water treatment requirements. Specifically, an ammonium ion selective electrode (ISE) may be used in the recirculating water (located in the sump or drain) to detect total NH₄⁺ in solution. A monitoring system and control loop may directly measure NH₄⁻ in solution and provide a millivolt signal back to a dual channel controller. The ISE may be connected to a controller (for example, Rosemount model 1056), which may supply a 4-20 mA signal to a pump controller. The pump controller may manage the pump frequency and duration for each component being fed into the air scrubber. This two channel controller may be configured to show ammonium concentration on one channel, and pH on the other, or may be connected to two independent ammonium ion ISE sensors. As such, components A and B may be fed into the air scrubber based on feedback from an ion selective electrode (ISE) monitoring and control feedback system. If the concentration of NH₃ high, then more of a composition comprising component A and B may be added to the air scrubber. If the concentration of NH₃ is low, then less of a composition comprising component A, B, and/or C may be added to the air scrubber. For example, about 250 mg/L of component A and about 75 mg/L of component B may be added to the air scrubber for ammonium levels of 1,000 mg/L in the recirculating water. If less ammonium is detected, for example, the amounts of components A and B may be reduced to at least about 125 and about 25 mg/L, respectively. The temperature of the inlet gas stream from the rendering plant may be less than 100° F., as hot gases may not dissolve into water and may therefore leave the scrubber as a malodor.

U.S. Pat. No. 4,206,074 issued Jun. 3, 1980, U.S. Pat. No. 4,780,237 issued Oct. 25, 1988, and EP 1,682,643 granted Aug. 22, 2007, each of which is hereby incorporated fully be reference, may each contain components that are useful in the methods and compositions for cleaning air scrubbers.

Embodiments of the invention are further detailed in the examples below.

### EXAMPLES

In the examples, mg/L refers to an amount of component being added in mg to a volume (L) of make up water in the air scrubber.

### Example 1

### Measuring Volatile Compounds in Air from an Air Scrubber

The following components were run through a high intensity air scrubber: 250 mg/L ReNew A (Diversey, Sturtevant, Wis.), 75 mg/L of a second component (Component B1 in Table 1 below), and citric acid added to a pH of 7.6. Each component was introduced directly into the air scrubber. The components were not premixed. This was found to be effective in removing VOCs from the air scrubber.

Compounds in an air sample exiting from the air scrubber were detected and quantified using GC-MS. The following compounds in the air sample were detected and quantified using GC-MS: 3-methyl-butanal; methylene chloride; 2-methyl-1-propene; 2-methyl-propanal; acetonitril SS Cyan; alpha,-diene SS Bi; methanethiol SS Merc; trimethylene oxide; penol SS Baker's P; acetaldehyde; 3-methyl-2-butanone; hexane; carbonyl sulfide SS, 1-propanol; 1-chloro-1,1-ethane; 2-methyl-butanal; 2-butanone; 1-butanol; ethanol; toluene; heptane; hydrochloric acid; hexanal; trichloroethylene; acetone; chloro-methane; 4-methyl-octane; 3-methyl-nonane; carbon disulfide; 1,2-dimethy-benzene; pentane SS n-pentane; trimethyl-silanol; 4-methyl-octane; ethanethiol; pentanal; and 1-(hexyloxy)-hexane. The amount of these compounds were compared to those from air exiting the same air scrubber run with conventional cleaners such as chlorine dioxide, acidified bleach, chlorine gas, and ozone.

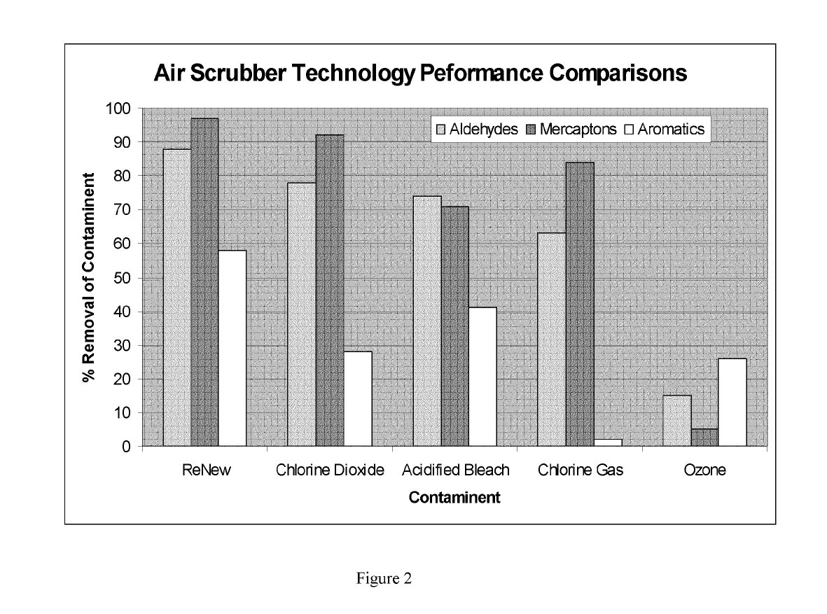

As shown in FIG. 2, a composition according to Example 1 (“Renew”) removed more aldehydes, mercaptans, and aromatics from air than conventional chlorine dioxide, acidified bleach, chlorine gas, and ozone.

### Examples 2-11

250 mg/L of ReNew A (Diversey, Sturtevant, Wis.) is run through a high intensity air scrubber with 75 mg/L of each of the various B components according to Table 1, along with citric acid being added in amounts bringing the pH between 7.0 and 8.0. The non-ionic surfactants identified in Table 1 are various Neodol™ ethoxylates, commercially available from Shell Chemicals (Houston, Tex.), and identified by trade name/number. The HLB values of each surfactant and each mixture are provided in the Table.

Example 2 includes 250 mg/L ReNew A, 75 mg/L of Component B-2, and citric acid added to a pH of 7.6. Example 3 includes 250 mg/L ReNew A, 75 mg/L of Component B-3, and citric acid added to a pH of 7.6. Example 4 includes 250 mg/L ReNew A, 75 mg/L of Component B-4, and citric acid added to a pH of 7.6 and so on. The components in the examples may be introduced directly and separately into the air scrubber. The components may also be premixed prior to introduction.

TABLE 1

| | | Component B | | | | | | | | | |
|---|---|---|---|---|---|---|---|---|---|---|---|
| non-ionic | | Ex. 2 | Ex. 3 | Ex. 4 | Ex. 5 | Ex. 6 | Ex. 7 | Ex. 8 | Ex. 9 | Ex. 10 | Ex. 11 |
| surfactant | HLB | B1 | B2 | B3 | B4 | B5 | B6 | B7 | B8 | B9 | B10 |
| Neodol ™ 91-2.5 | 8.1 | 25% | | | 10% | | | | | 40% | |
| Neodol ™ 91-5 | 11.6 | | | | | | | | | | |
| Neodol ™ 91-6 | 12.5 | 75% | | | | | | | | | |
| Neodol ™ 91-8 | 13.7 | | | | | | | | | 60% | |
| Neodol ™ 45-4 | 8.9 | | | | | 10% | | | | | |
| Neodol ™ 45-7 | 11.8 | | | | 90% | 90% | 90% | | | | |
| Neodol ™ 23-3 | 7.9 | | | 30% | | | 10% | | | | |
| Neodol ™ 23-6.5 | 11.8 | | 100% | | | | | | | | |
| Neodol ™ 25-3 | 7.5 | | | | | | | 40% | 35% | | |
| Neodol ™ 25-5 | 10.3 | | | | | | | | | | 50% |
| Neodol ™ 25-7 | 12.2 | | | | | | | | | | 50% |
| Neodol ™ 25-9 | 13.2 | | | 70% | | | | | 65% | | |
| Neodol ™ 25-12 | 14.4 | | | | | | | 60% | | | |
| total | | 11.4 | 11.8 | 11.61 | 11.43 | 11.51 | 11.41 | 11.64 | 11.21 | 11.46 | 11.25 |
| HLB | | | | | | | | | | | |

### Example 12

### Measuring Volatile Compounds in Air from an Air Scrubber

The following components were run through a high intensity air scrubber: 250 mg/L ReNew A (Diversey, Sturtevant, Wis.), 75 mg/L of Tergitol 15-S-7 (Dow, Midland, Mich.), and citric acid added to a pH of 7.6. Each component was introduced directly into the air scrubber. The components were not premixed.

### Example 13

### Optimal pH to Remove ammonia and Hydrogen Sulfide

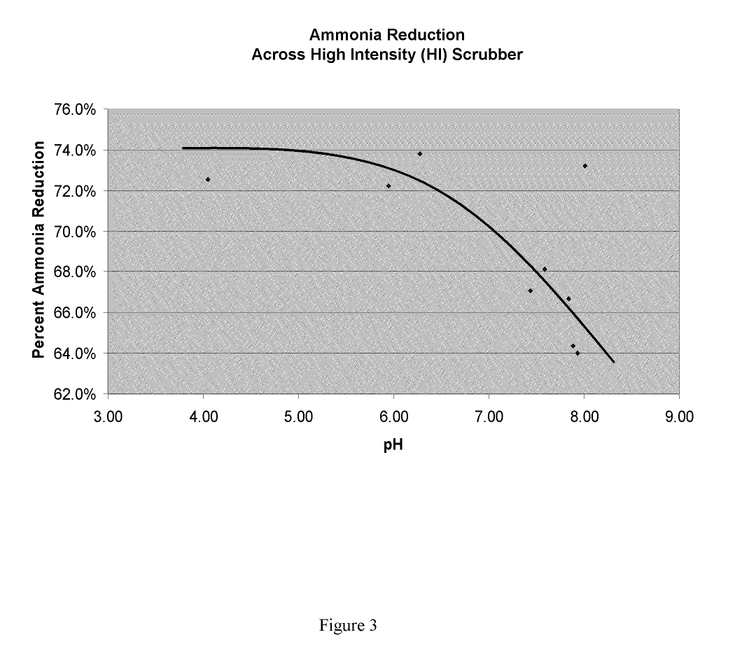

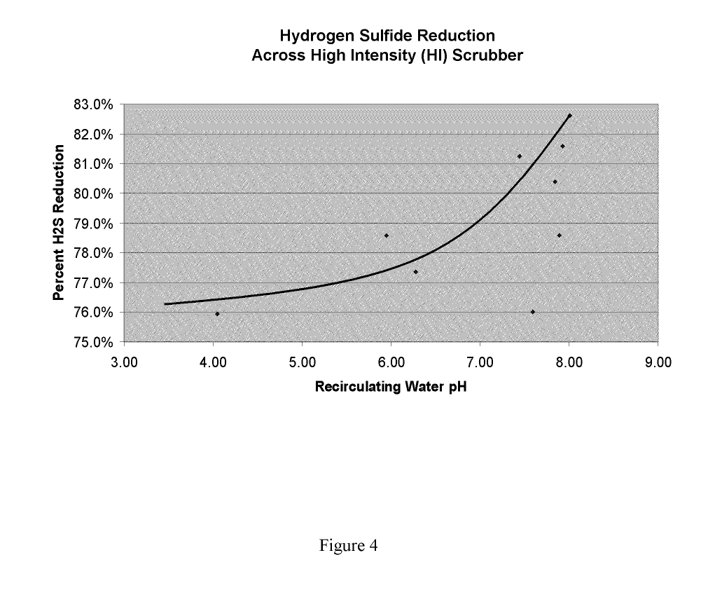

Ammonia and hydrogen sulfide were measured in air exiting from an air scrubber using Example 1. The pH of the composition was monitored. As shown in FIG. 3, a lower pH yielded a greater reduction in ammonia from the air. As shown in FIG. 4, more hydrogen sulfide was eliminated from the air at a greater pH of the composition.

### Example 14

### Cleaning Performance of Low Foaming Surfactants

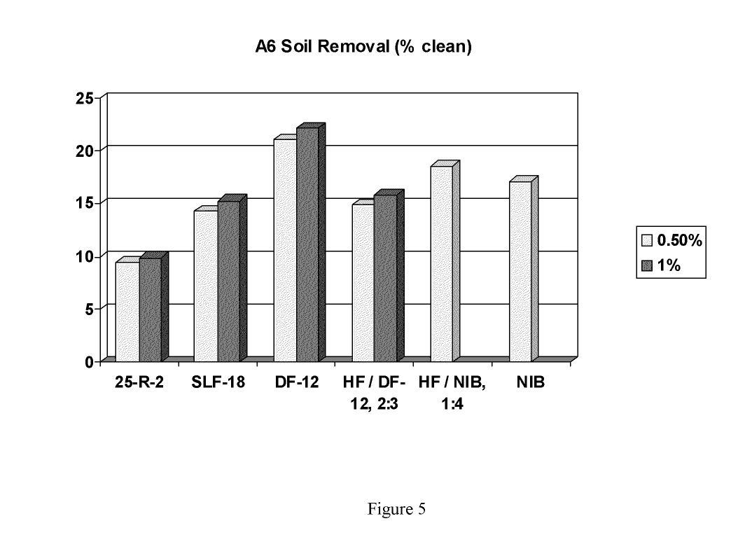

The following low foaming surfactants were tested for cleaning performance using A6 soiling method of the ASTM D4488 standard cleaning method: Pluronic® 25-R-2 (100%), Pluronic® SLF-18 (100%), Triton™ DF-12 (100%), “High Foam” and Triton™ DF-12 mixed in a ratio of 2:3, “High Foam” and NIB mixed in a ratio of 1:4, and NIB. “High Foam” (HF) was a mixture of 70% cocobetaine (35%) and 30% cocamidopropyl betaine (42%). NIB (non-ionic blend) was a hard surface cleaning standard comprising a 2:1 blend of Neodol™ 91-6 and Neodol™ 91-2.5. As shown in FIG. 5, Triton™ DF-12 removed more soil than the other compositions. Tests were done at 0.5% and 1.0% surfactant (balance water).

### Example 15

### Foam Profile.

Foaming of various compositions, detailed in Table 2, was determined. Foaming was determined by re-circulating 5 liters of a composition in an Autochlor low temperature dish machine for a 1 minute interval and a 5 minute interval. The foam height above the water level in the sump was measured immediately after the machine was stopped and also 1 minute later. In Table 2, “High Foam” was 70% cocobetaine (35%) and 30% cocoamidopropyl betaine (42%), and OT indicates the foam was over the top of the sump.

TABLE 2

| | | Foam Height | | | |
|---|---|---|---|---|---|
| Composition | Temp | T1 = 0 min | T2 = 1 min | T5 = 0 min | T5 = 1 min |

Triton ™ DF-12

| 50 ppm High Foam | 40° C. | O.T. | | O.T | | | | | |
|---|---|---|---|---|---|---|---|---|---|
| 50 ppm High Foam, | 40° C. | 0.15 | cm | 0 | cm | 3.5 | cm | 0.25 | cm |
| 60 ppm Triton ™ DF-12 | | | | | | | | | |
| 50 ppm High Foam, | 30° C. | 0.2 | cm | 0 | cm | 1.5 | cm | 0.2 | cm |
| 60 ppm Triton ™ DF-12 | | | | | | | | | |
| 50 ppm High Foam, | 20° C. | 1.5 | cm | 0.2 | cm | 2.5 | cm | 0.2 | cm |

| 60 ppm Triton ™ DF-12 | | | | | | | |
|---|---|---|---|---|---|---|---|
| 50 ppm High Foam, | 12° C. | OT | 4.5 | cm | OT | 5.5 | cm |

60 ppm Triton ™ DF-12

Plurafac ® LF-403

| 50 ppm High Foam | 40° C. | O.T. | | O.T | | | | | |
|---|---|---|---|---|---|---|---|---|---|
| 50 ppm High Foam, | 40° C. | 0.15 | cm | 0 | cm | 3.5 | cm | 0.25 | cm |
| 60 ppm Plurafac ® LF-403 | | | | | | | | | |
| 50 ppm High Foam, | 30° C. | 4.0 | cm | 0.2 | cm | 4.0 | cm | 0.3 | cm |
| 60 ppm Plurafac ® LF-403 | | | | | | | | | |
| 50 ppm High Foam, | 20° C. | 4.0 | cm | 0.5 | cm | 4.0 | cm | 0.3 | cm |

| 60 ppm Plurafac ® LF-403 | | | | | | | | |
|---|---|---|---|---|---|---|---|---|
| 50 ppm High Foam, | 12° C. | 5.5 | cm | 3.5 | cm | OT | 5.5 | cm |

60 ppm Plurafac ® LF-403

Macol LF-101

| 50 ppm High Foam | 40° C. | O.T. | | O.T | | | | | |
|---|---|---|---|---|---|---|---|---|---|
| 50 ppm High Foam, | 40° C. | 0.2 | cm | 0 | cm | 0.2 | cm | 0 | cm |
| 60 ppm Macol LF-101 | | | | | | | | | |
| 50 ppm High Foam, | 20° C. | 2.5 | cm | 0.2 | cm | 3.0 | cm | 0.3 | cm |
| 60 ppm Macol LF-101 | | | | | | | | | |
| 50 ppm High Foam, | 20° C. | 6.5 | cm | 2.0 | cm | 10.0 | cm | 3.0 | cm |

60 ppm Macol LF-101

Pluronic ® N-3

| 50 ppm High Foam | 40° C. | O.T. | | O.T | | | | | |
|---|---|---|---|---|---|---|---|---|---|
| 50 ppm High Foam, | 40° C. | 0 | cm | 0 | cm | 0 | cm | 0 | cm |
| 60 ppm Pluronic ® N-3 | | | | | | | | | |
| 50 ppm High Foam, | 40° C. | 2.5 | cm | 0 | cm | 5 | cm | 0 | cm |

| 60 ppm Pluronic ® N-3 | | | | | | |
|---|---|---|---|---|---|---|
| 50 ppm High Foam, | 30° C. | 5.0 | cm | 1 | cm | OT |
| 60 ppm Pluronic ® N-3 | | | | | | |
| 50 ppm High Foam, | 20° C. | 6.0 | cm | 5 | cm | OT |

60 ppm Pluronic ® N-3

Others

| 50 ppm High Foam | 40° C. | O.T. | | O.T | | |
|---|---|---|---|---|---|---|
| 50 ppm High Foam, | 30° C. | 5.0 | cm | 2.0 | cm | |
| 60 ppm Pluronic ® 25-R-2 | | | | | | |
| 50 ppm High Foam, | 30° C. | OT | | OT | | |
| 60 ppm Plurafac ® L-62 | | | | | | |
| 50 ppm High Foam, | 30° C. | 6.0 | cm | 3.0 | cm | OT |

60 ppm Triton ™ CF-10

Compositions without “High Foam” Component

| 60 ppm Pluronic ® L-62 | 12° C. | 4.5 | cm | 1.0 | cm | 5.0 | cm | 1.5 | cm |
|---|---|---|---|---|---|---|---|---|---|
| 120 ppm Pluronic ® L-62 | 12° C. | | | | | 5.5 | cm | 2.0 | cm |
| 60 ppm Pluronic ® SLF-18 | 12° C. | 4.0 | cm | 0 | cm | 6.5 | cm | 0 | cm |
| 120 ppm Pluronic ® SLF-18 | 12° C. | | | | | 6.0 | cm | 0 | cm |
| 60 ppm Plurafac ® LF-403 | 12° C. | 2.0 | cm | .2 | cm | 4.5 | cm | 1.0 | cm |
| 120 ppm Plurafac ® LF-403 | 12° C. | 2.0 | cm | .2 | cm | 4.5 | cm | 1.0 | cm |
| 60 ppm Triton ™ DF-12 | 12° C. | 0 | cm | 0 | cm | 0 | cm | 0 | cm |
| 120 ppm Triton ™ DF-12 | 12° C. | 0 | cm | 0 | cm | 0 | cm | 0 | cm |
| 60 ppm Pluronic ® 25-R-2 | 12° C. | 3.5 | cm | 0 | cm | 4.0 | cm | 0 | cm |

| | | | | | |
|---|---|---|---|---|---|
| 120 ppm Pluronic ® 25-R-2 | 12° C. | 4.5 | cm | 0 | cm |

| | | | | | | | |
|---|---|---|---|---|---|---|---|
| 60 ppm NIB (non-ionic | 12° C. | 9.5 | cm | 9.5 | cm | OT | OT |

blend: 2:1 blend of
Neodol ™ 91-6 and
Neodol ™ 91-2.5)

From the results in Table 2, the surfactants listed in order from lowest foaming to highest foaming are the following: Triton™ DF-12, Plurafac® LF-403, Pluronic® 25-R-2, Pluronic® N-3, Pluronic® SLF-18, Triton™ CF-10, Pluronic® L-62, and NIB.

### Example 16

Each of the surfactants in Example 14 and 15 (e.g., Pluronic® 25-R-2 (100%), Pluronic® SLF-18 (100%), Triton™ DF-12 (100%), “High Foam” and Triton™ DF-12 mixed in a ratio of 2:3, “High Foam” and NIB mixed in a ratio of 1:4, and NIB) is separately used in a commercial air scrubber. In separate tests, each surfactant is introduced directly into and run through a commercial air scrubber. For example, Triton™ DF-12 (100%) is introduced directly into and run through a commercial air scrubber at 120 ppm for a period of time. It is expected to clean the air scrubber and/or reduce malodors. These properties are monitored over 4 hour periods, and after two weeks. Each surfactant is generally run at 100-150 ppm, and particularly, at 120 ppm.

Plurafac® LF-403 (100%) may be introduced directly into and run through a commercial air scrubber at 120 ppm for a period of time. It is expected to clean the air scrubber and/or reduce malodors.

Each surfactant from Example 14, as well as Plurafac® LF-403 (100%), may also be used in combination with ReNew A (Diversey, Sturtevant, Wis.), in which case 80 ppm of the component is run with 250 ppm of ReNew A for a period of time. This is expected to clean the air scrubber and/or reduce malodors.

Measuring a decrease in pressure drop in the air scrubber means the composition is effective in cleaning the air scrubber. The compositions are also expected to be relatively low foaming. Foam height will be reported at cm of foam above water level. Pressure differential, malador, foam height and/or cleanability may be measured on an hourly basis during first day, and every 2-4 hours thereafter.

### Example 17

Foam Profiled of Low-Foam Surfactants

Foaming was determined by re-circulating 5 liters of a surfactant solution in an Autochlor low temperature dish machine for a 1 minute interval and a 5 minute interval. The foam height above the water level in the sump was measured immediately after the machine was stopped and 1 minute later. Briefly, an Autochlor Model A4 Dish Machine, 100 mL plastic beaker, and Mettler PM4800 balance were used. A plastic ruler was positioned and secured in the sump of machine such that the ruler touched the bottom of the sump. The machine was rinsed by filling the machine with fresh tap water, recirculating for 1 minute, and draining. This rinsing was repeated three times or until no foam was present in the sump of the machine. In a pail, 5 L of water were measured of desired temperature and added to the wash machine. The temperature and height of water in sump were recorded. The water was recirculated and water pressure was recorded. Once the water recirculation was stopped, test products were added to 50 mL of tap water in a 100 mL beaker by weighing on a balance and then mixing. It was then poured into the sump of wash machine, and the 100 mL beaker was rinsed with sump water to ensure complete transfer of product. The water was recirculated in the wash machine for 1 minute. The foam height above the water level in the sump was recorded at 0 and 1 minute. If foam spilled out of sump, it was recorded as “over the top” or “OT”. Water was then recirculated in the wash machine for 5 minutes, and then the recirculation was turned off. The foam height above the water level in the sump was recorded at 0 and 1 minute. If foam spilled out of sump, it was record as “over the top” or “OT”. The machine was drained and rinsed.

Low foam surfactants were selected based on two criteria: estimated ability to clean, and anticipated low foaming characteristics. A standard cleaning nonionic (Neodol™ 23-6.5) was also selected in order to use as a reference and to blend with the low foam surfactants to understand if blended materials would have the desired cleaning/foaming profile. The selected surfactants fell primarily into the category of alkoxylated alcohols, indicated below, with the cloud point of a 1% solution in parentheses:
Plurafac® (fatty alcohol alkoxylate)→LF-400 (33° C.), LF-403 (41° C.), LF-101 (12° C.)
Pluronic® (EO/PO block copolymer)→25-R-2 (29° C.), N-3 (30° C.), L-61(17° C.), L-62 (24° C.)
Triton™ (capped alkoxylates)→CF-10 (28° C.), CF-32 (25° C.), DF-12 (17° C.), SLF-18 (19° C.)
Neodol™ (linear alcohol ethoxylate)→23-6.5 (41° C.)

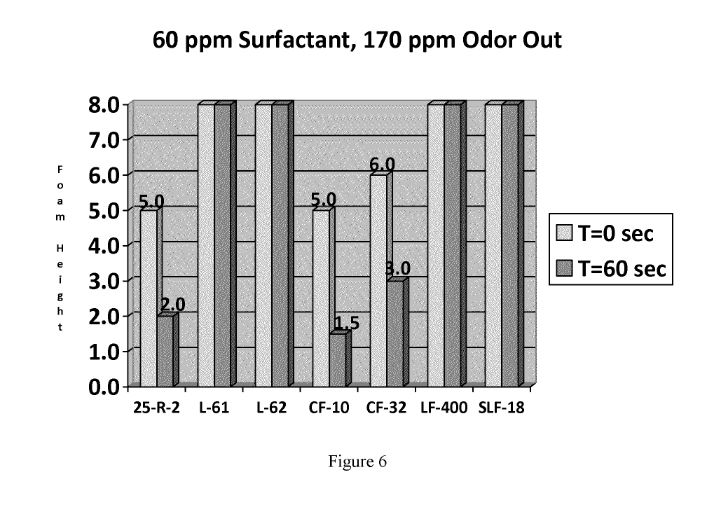

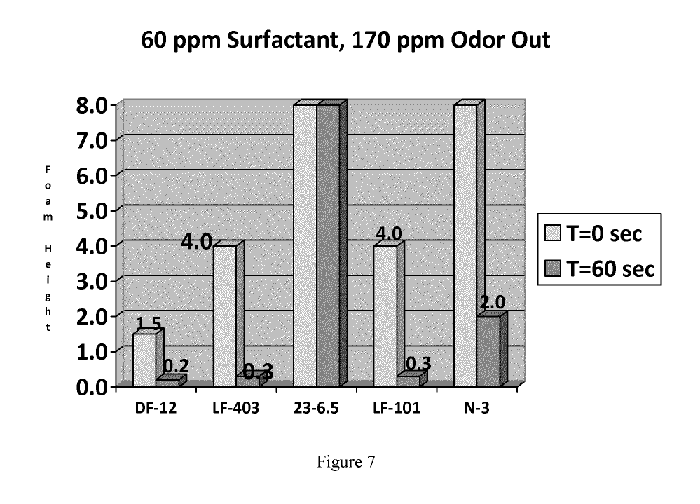

The selected surfactants were combined with ReNew A (00) to final concentrations of 60 ppm surfactant and 170 ppm ReNew A. Neodol™ 23-6.5 (ASCB) was used a non-low foam surfactant control. Results are shown in FIGS. 6 and 7, wherein 8 cm indicated was the top of the sump and represented an overflow of foam out of the sump.

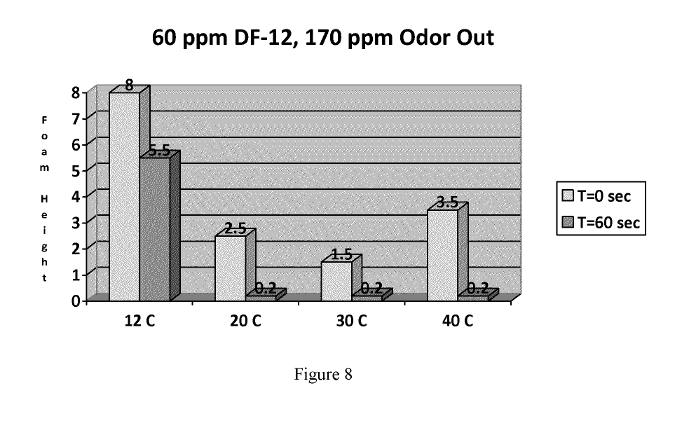

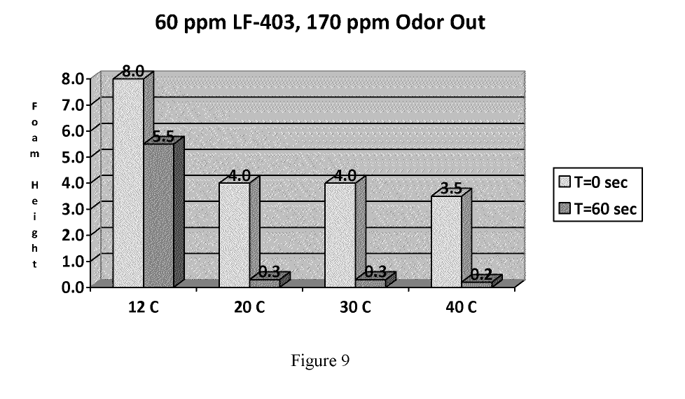

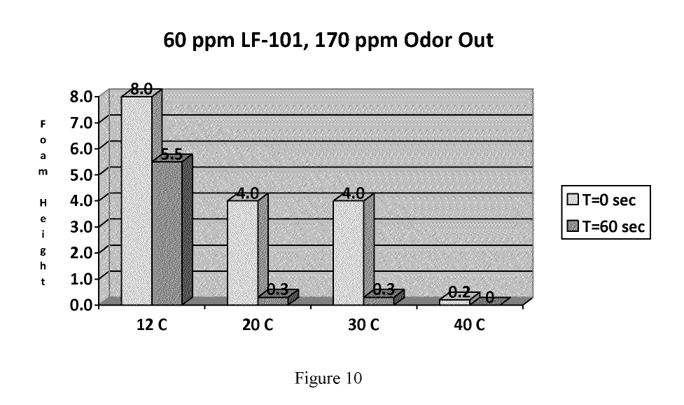

The best performing low foam surfactant was Triton™ DF-12, followed by Plurafac® LF-403 and Plurafac® LF-101 (FIGS. 8, 9, and 10). Initial foam height was well contained and did not build over time. The foam was not stable as demonstrated by the quick collapse seen at 1 minute after stopping recirculation. These were a marked improvement versus Neodol™ 23-6.5.

This group was further test over the temperature range that air scrubbers could be operated, including 12° C. Given that the cloud point of Triton™ DF-12 is 17° C., it was not surprising that foam increased significantly at 12° C. It did take the full 5 minutes of recirculation for the foam to eventually build to overflow, and it did continue to collapse over time with complete collapse taking about 3 minutes after cessation of recirculation. Plurafac® LF-403 performed well and similar to Triton™ DF-12, except at 12° C. where foam became a big problem. If air scrubber temperature could be maintained at 20° C. or higher, Plurafac® LF-403 may be a good option for foam control. Plurafac® LF-101 had superior performance at 40° C. with essentially no foam formed at all. The performance of all three was considered acceptable from a foaming perspective for use as a low foam surfactant.

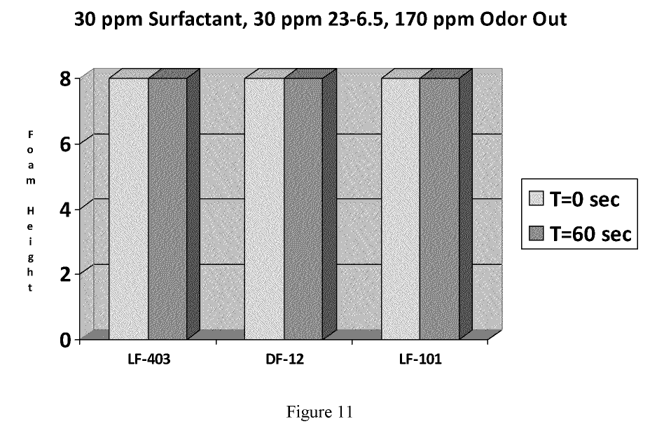

Next, Triton™ DF-12, Plurafac® LF-403, and Plurafac® LF-101 were tested to see if they could control foam when mixed in equal parts with Neodol™ 23-6.5. Results are shown in FIG. 11 (ASCB, Neodol™ 23-6.5). None was able to control foam when mixed in equal parts with Neodol™ 23-6.5, even at the relatively high temperature of 30° C.

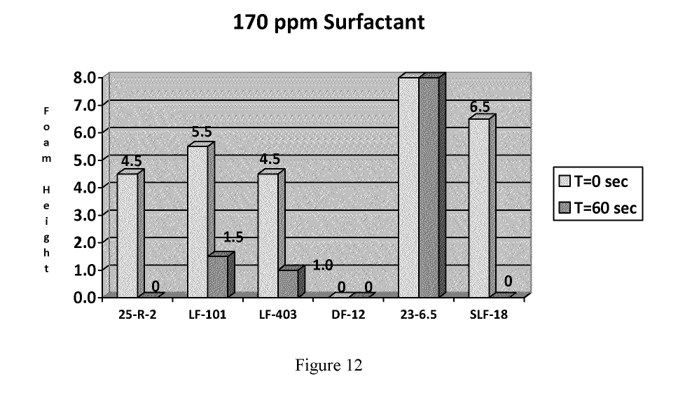

The foam properties of the low foam surfactant alone were examined next, and results are shown in FIG. 12 (ASCB, Neodol™ 23-6.5). Since the most difficult temperature to control foam was at 12° C., this was used screen the low foam set. Triton™ DF-12, Plurafac® LF-403, and Plurafac® LF-101 were used in addition to three other candidates that were anticipated to be good cleaning surfactants. Triton™ DF-12 provided superior foam control. Pluronic® 25-R-2 and Triton™ SLF-18 had foam that collapsed very rapidly after agitation was stopped. The overall rank of the screened low foam surfactants, in terms of foam, was as shown in Table 3.

TABLE 3
Foam Ranking.

| | |
|---|---|
| Triton ™ DF-12 | 1 |
| Plurafac ® LF-403 | 2-3 |
| Plurafac ® LF-101 | 2-3 |
| Pluronic ® 25-R-2 | 4 |
| Triton ™ CF-10 | 5 |
| Triton ™ CF-32 | 6 |
| Pluronic ® N-3 | 7 |
| Pluronic ® SLF-18 | 8 |
| Pluronic ® L-61 | 9 |
| Pluronic ® L-62 | 10 |
| Plurafac ® LF-400 | 11 |
| Neodol ™ 23-6.5 | 12 |

Cleaning Ability of Low-Foam Surfactants

The A6 soiling method of the ASTM D4488 standard cleaning method was used to determine cleaning performance of various surfactant systems. Briefly, Armstrong vinyl composition tiles were soiled with a mixture of 50 parts Stoddard solvent, 10 parts mineral oil, 4 parts vegetable oil, 4.5 parts carbon black, and 10 parts black charm clay. Tiles were baked at 50° C. for one hour and cleaned on a Precision Force Applicator In-Line Tester. Soil removal was determined with a Minolta CR-410 Chroma-meter. To prepare A6 soil, 50 parts Stoddard solvent, 10 parts mineral oil, 4 parts vegetable oil, 4.5 parts carbon black, and 10 parts black charm clay were mixed. Parts were mixed by weight and came to a total of 78.5 parts. It was mixed overnight with a magnetic stirrer and then for at least two minutes prior to use. To apply the soil, the tiles were laid on a bench with the back side facing up and the grain in the same direction. An extra tile for priming the brush was included. The soil was mixed continuously until all tiles were soiled. First, the extra tile was soiled to prime the brush. This tile was then discarded. 3.00 mL of soil was applied using the Fisherbrand® Finnpipette® 10 mL pipetter, with a new tip used for each 12″×12″ tile. The soil was applied over the entire back of the tile (starting in the center and drawing a large spiral) to cover as much of the tile as possible. A two-inch nylon brush was used to spread soil evenly over the tile. The bristles were soaked in mineral spirits, and the brush was blotted on a paper towel before using to allow the soil to spread smoothly and easily. The soil was spread over the whole tile vertically, then horizontally, continuing until the tile was coated uniformly and completely. The grain of the tile was no longer visible, but its direction was known since the tiles were laid out with the grain in the same direction. The soil was dried for 20 minutes at room temperature. The tiles were laid in a 50° C. oven to cure for one hour. The tiles were removed promptly and allowed to cool at room temperature for at least 20 minutes on a flat surface. For a pre-test preparation, a Minolta Chroma-meter was calibrated using a white calibration plate for the CR-410 (No. 20733134). Tiles were cut into 4″x 12″ strips so that the length of the tile was with the grain of the tile (seen by the brush strokes). This was so that all cleaning would be done with the grain. Each strip was labeled and read nine times using the PMMA reading template. Cleaning samples were diluted to desired concentration in tap water at room temperature. Fleece was cut into 4″×12″ strips (one strip of fleece for each tile strip). To clean a soiled VCT tile, the PAI program and PFA In-Line Tester were used. A piece of cut fleece (4″×12″) was wrapped around plastic block lengthwise and placed into a PFA block holder. A metal tray with PMMA template was placed into a tray holder on PFA machine and a 4″×12″ soiled tile was placed into a plastic template. A new piece of fleece was used for each tile strip. 200 g of diluted cleaner was poured into a tray containing the soiled tile so the tile was completely covered by cleaning solution. The tile was allowed to soak for 30 seconds. The template was pushed down to expel air bubbles so the block would run over the tile and template smoothly. When there was approximately 10 seconds of soak time left, the pressure was adjusted to 5 pounds by turning the top wheel on the PFA machine. When the soak time was over, 10 cycles were then completed. The tile strip was removed from the metal tray and rinsed with cold water. The tile was allowed to air dry. The metal tray, PMMA template, and block were rinsed before continuing to the next sample. Once all samples were run and dry, each tile strip was re-read using the Minolta CR-410 and the PMMA reading template. The soiled readings (Yₛ) and cleaned readings (Y_{c}) were recorded, and the percent soil removed was determined using the equation below:

$$\% soil removed = \frac{( {Y}_{c} - {Y}_{s} )}{( {Y}_{o} - {Y}_{s} )}$$

$$where$$

$${Y}_{o} is 94.80 . ( {Y}_{o} is the value of the calibration plate . )$$

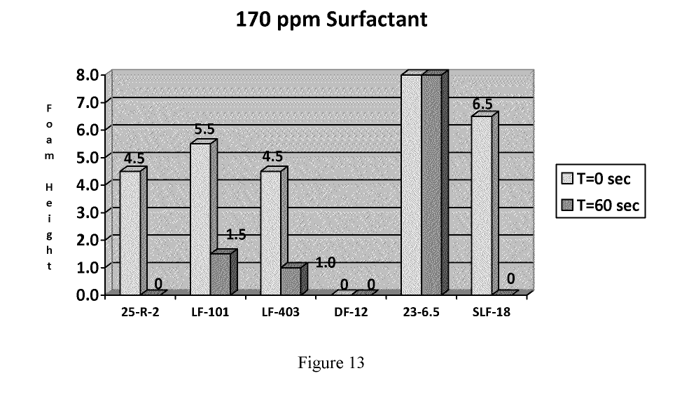

The best candidates from the foaming tests described above were screened for cleaning performance. These were compared to Neodol™ 23-6.5, and the composition of 170 ppm ReNew A and 80 ppm Neodol™ 23-6.5, scaled to 0.5% total surfactant. This was done because a 100% level would not show any difference on the A6 soil removal test. The low foam surfactants were run at 0.5% and 1% to make sure suitable ranges were covered. Results are shown in FIG. 13 (00, ReNew A; ASCB, Neodol™ 23-6.5).

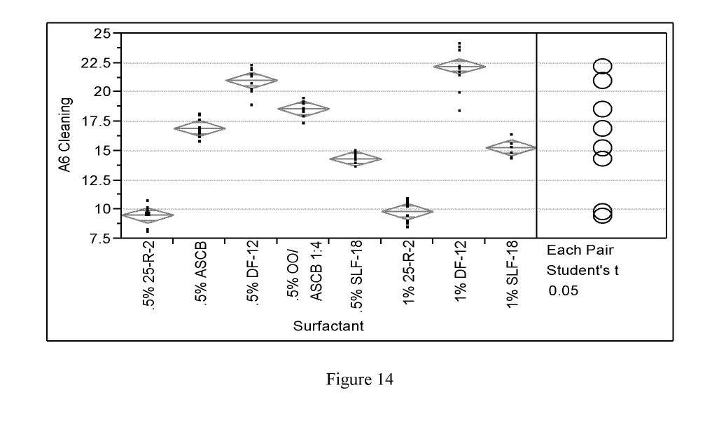

The two dilutions of Triton™ DF-12 (0.5% and 1%) provided statistically better cleaning than all other systems (FIG. 14), including the 170 ppm ReNew A and 80 ppm Neodol™ 23-6.5 and Neodol™ 23-6.5 by itself. Triton™ DF-12 clearly out performed the other two low foam surfactants (Pluronic® 25-R-2 and Triton™ SLF-18) by a wide margin.

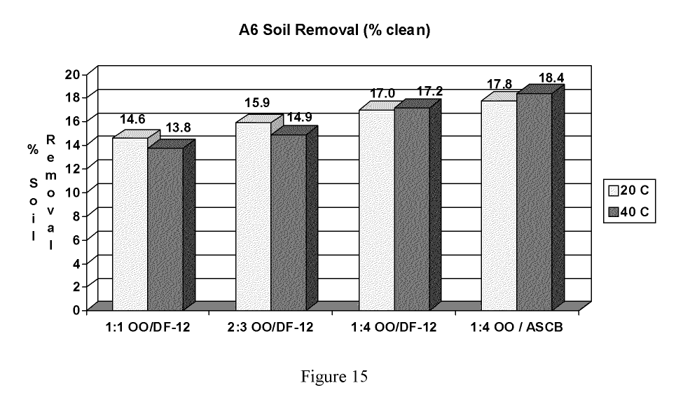

The next step was to look at how Triton™ DF-12 performed when combined with ReNew A. The cloud point of Triton™ DF-12 is 17° C. Cleaning performance may be compromised if the temperature moved too far above cloud point. Therefore, this round of test was performed at a fixed concentration of 0.7% active surfactant and two different temperatures; 20° C. and 40° C., the nominal and high end of the air scrubber operating temperature. Results are shown in FIG. 15 (OO, ReNew A; ASCB, Neodol™ 23-6.5).

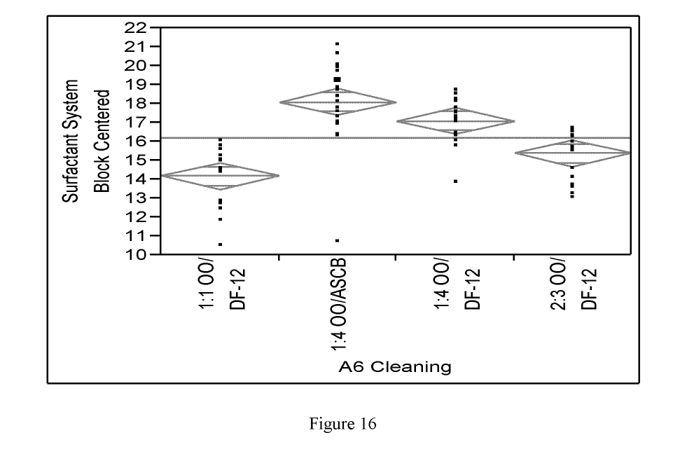

The 1:4 ReNew A/Neodol™ 23-6.5 system had statistically similar performance to the ReNew A/Triton™ DF-12 combination (FIG. 16). The 1:4 ReNew A/Triton™ DF-12 system also had the best performance of the three ratios of Triton™ DF-12 and ReNew A tested.

In lab tests, Triton™ DF-12 demonstrated the best foam control over a 12° C. to 40° C. temperature range both with and without ReNew A. None of the low foam surfactants tested were able to control foam when a standard nonionic, such as a linear alcohol ethoxylate, was added. The next best option tested was Plurafac® LF-403.

Cleaning performance tests showed that Triton™ DF-12 had superior performance to all other low foam surfactants tested. Further testing of Triton™ DF-12 indicated that it achieved optimal performance when paired with Renew A at a 1:4 active surfactant ratio.

### Example 18

A composition comprising 170 ppm ReNew A (Diversey, Sturtevant, Wis.) and 80 ppm Triton™ DF-12 was used in at least one commercially-available wet air scrubber for approximately 2-4 months. Citric acid was used to maintain the pH of the composition in the air scrubber to approximately pH 7.6, and the temperature was maintained below approximately 40° C. A satisfactory foam level of low to no foam was maintained.

Thus, the invention provides, among other things, a composition for cleaning and operating wet air scrubbers.

1. A method of operating a wet air scrubber, the method comprising:
(a) adding to the wet air scrubber a composition comprising water and at least one of Component A and Component B, wherein
(i) Component A comprises:
(1) an amphoteric surfactant; and
(2) an enzyme;
(ii) Component B comprises at least one of:
(1) a detersive surfactant and a defoamer;
(2) at least one low-foam surfactant selected from a capped alkoxylate, an EO/PO (ethylene oxide group/propylene oxide group) block copolymer, a fatty alcohol alkoxylate, a nonionic ethylene oxide (EO) containing surfactant, an alkoxylated amine, an amine surfactant, a low cloud point nonionic surfactant, and a combination thereof; and
(3) a combination of (ii)(1) and (ii)(2);
(b) adding a pH control agent to the composition to maintain a pH of about 6 to about 9; and
(c) maintaining the water temperature at less than about 50° C.

2. The method of claim 1, wherein the composition comprises at least one of a capped alkoxylate, an EO/PO (ethylene oxide group/propylene oxide group) block copolymer, and a fatty alcohol alkoxylate.

3. The method of claim 1, wherein the pH control agent comprises an acid.

4. The method of claim 3, wherein the acid comprises citric acid.

5. The method of claim 1, wherein the composition comprises Component A.

6. The method of claim 1, wherein the composition comprises Component A and the surfactant comprises a zwitterion.

7. The method of claim 1, wherein the composition comprises Component A, and the surfactant of Component A comprises two amphoteric surfactants at about 5 to 15% wt of Component A.

8. The method of claim 1, wherein the composition comprises Component A, and the enzyme comprises at least one of an amylase, a protease, and a lipase.

9. The method of claim 1, wherein the composition comprises Component A and Component B.

10. The method of claim 9, wherein Component A and Component B are added to a combined concentration of about 1 ppm to about 400 ppm in the wet air scrubber.

11. The method of claim 9, wherein Component A and Component B are present in the wet air scrubber at a ratio of about 1:2 to about 1:6.

12. The method of claim 9, wherein Component A and Component B are present in the wet air scrubber at a ratio of about 1:4.

13. The method of claim 1, wherein the composition comprises Component B.

14. The method of claim 13, wherein Component B comprises a surfactant having an HLB of about 8 to about 14.

15. The method of claim 1, wherein the composition comprises Component B(1).

16. The method of claim 15, wherein Component B(1) comprises at least one of a primary alcohol ethoxylate and a secondary alcohol ethoxylate combined in a ratio such that the HLB is about 8 to about 14.

17. The method of claim 1, wherein the composition comprises Component B(2).

18. The method of claim 17, wherein Component B(2) comprises at least one of a capped alkoxylate, an EO/PO (ethylene oxide group/propylene oxide group) block copolymer, a fatty alcohol alkoxylate, and a combination thereof.

19. The method of claim 1, wherein the composition comprises a defoamer.

20. The method of claim 1, further comprising defoaming the composition with a mechanical defoamer.

21. The method of claim 1, wherein the composition comprises a detersive surfactant.

22. The method of claim 1, wherein the composition comprises at least one of a capped ethoxylate; an EO/PO (ethylene oxide group/propylene oxide group) block copolymer; an ethylene oxide derivative; an alcohol ethoxylate; and a low-foam surfactant.

23. The method of claim 1, wherein the composition comprises Component B, and Component B comprises a capped alkoxylate.

24. The method of claim 1, wherein the composition comprises Component B, and Component B comprises an EO/PO (ethylene oxide group/propylene oxide group) block copolymer.

25. The method of claim 1, wherein the composition comprises Component B, and Component B comprises a fatty alcohol alkoxylate.

26. The method of claim 1, wherein the composition comprises Component B, and Component B comprises an alcohol ethoxylate.

27. The method of claim 1, wherein the composition comprises Component B, and Component B comprises a capped alcohol ethoxylate.
